(19) United States
(12) Patent Application Publication
(10) Pub. No.: US 2025/0388847 A1
(43) Pub. Date: Dec. 25, 2025

(54) Stirring Method And Stirring Device

(71) Applicant: Terumo Kabushiki Kaisha, Tokyo (JP)

(72) Inventors: Liming LIU, Kanagawa (JP); Itaru Okubo, Kanagawa (JP)

(21) Appl. No.: 19/310,807

(22) Filed: Aug. 26, 2025

Related U.S. Application Data
(63) Continuation of application No. PCT/JP2024/007196, filed on Feb. 28, 2024.

(30) Foreign Application Priority Data
Feb. 28, 2023 (JP) 2023-0029017

(51) Int. Cl. C12M 1/33; C12M 1/34; C12M 1/36

(52) U.S. Cl. CPC C12M 45/02; C12M 1/3453; C12M 41/48

(57) ABSTRACT

A stirring method includes: stirring cell suspension by circulating the cell suspension in a circulation path including a bioreactor; acquiring a predetermined or selected physical quantity related to the cell suspension at a measurement target site in the circulation path over time; and determining if a uniform diffusion state in which cells are uniformly diffused in the cell suspension is reached by comparing a degree of variation of the physical quantity to the a predetermined or selected degree.

### CROSS-REFERENCE TO RELATED APPLICATIONS

The present application is a continuation of the International Patent Application No. PCT/JP2024/007196 filed Feb. 28, 2024, which designated the U.S. and claims the benefit of priority from Japanese Patent Application No. JP20230029017 filed Feb. 28, 2023. The entire disclosures of the above-identified applications are incorporated herein by reference.

### FIELD

The present invention relates to a stirring method and a stirring apparatus for stirring a cell suspension.

### BACKGROUND

Cell suspensions should be stirred during cell culture to efficiently culture cells. Stirring the cell suspension, allows all cells in the suspension to be appropriately nourished. Japanese Patent Application Laid-Open No. 2002-148258 discloses, for example, a ventilation stirring type culture apparatus.

In cell culture processing, a series of steps, including cell stirring and cell culturing, are repeatedly performed, for example, about every 24 hours. Optimizing cell stirring step is an important step in more broadly optimizing cell culture processing.

### SUMMARY

At least one example embodiment relates to a stirring method for a cell suspension. In at least one example embodiment, the stirring method may include stirring a cell suspension by circulating the cell suspension in a circulation path, acquiring a predetermined or selected physical quantity related to the cell suspension at a measurement target site in the circulation path over time, and determining if a uniform diffusion state has been reached. The circulation path may include a bioreactor. The uniform diffusion state refers to a state in which cells are uniformly diffused in the cell suspension. The uniform diffusion state may be reached when a degree of variation of the predetermined or selected physical quantity is smaller than a predetermined or selected degree.

While determining if the uniform diffusion state has been reached, the method may include comparing the degree of variation with a predetermined or selected degree. By monitoring the predetermined or selected physical quantity and determining if the uniform diffusion state has been reached or not, the cell stirring step can be performed only for a necessary minimum time to achieve the uniform diffusion state. Accordingly, the time required for the cell stirring, and in turn the time for the cell culture processing, can be optimized.

In at least one example embodiment, the degree of variation of the physical quantity may be a standard deviation of a plurality of the physical quantity within a predetermined or selected unit of time.

In at least one example embodiment, the physical quantity may be acquired using an optical sensor.

In at least one example embodiment, the physical quantity may be at least one of an amount of transmitted light transmitted through the cell suspension and an amount of scattered light scattered by the cell suspension.

In at least one example embodiment, the stirring of the cell suspension may be stopped when the uniform diffusion state has been reached.

In at least one example embodiment, the cell suspension may be stirred by rotating the bioreactor using an actuator.

In at least at least one example embodiment, the cell suspension may be brought into a uniform diffusion state more quickly by stirring the cell suspension by rotation the bioreactor.

At least one example embodiment relates to a stirring apparatus. In at least one example embodiment, the stirring apparatus may include a control unit that may be configured to stir a cell suspension by circulating the cell suspension in a circulation path; an acquisition unit that may be configured to acquire a predetermined or selected physical quantity related to the cell suspension at a measurement target site in the circulation path over time; and a determination unit that may be configured to determine if a uniform diffusion state in which cells are uniformly diffused in the cell suspension has been reached. The uniform diffusion state may be reached when t a degree of variation of the physical quantity is smaller than a predetermined or selected degree. The circulation path may include a bioreactor.

In at least one example embodiment, the degree of variation of the physical quantity may be a standard deviation of a plurality of the physical quantity within a predetermined or selected unit of time.

In at least one example embodiment, the physical quantity may be acquired using an optical sensor.

In at least one example embodiment, the physical quantity may be at least one of an amount of transmitted light transmitted through the cell suspension and an amount of scattered light scattered by the cell suspension.

In at least one example embodiment, the control unit may be configured to stop stirring of the cell suspension when the uniform diffusion state has been reached.

In at least one example embodiment, the control unit may be configured to stir the cell suspension by rotating the bioreactor using an actuator.

### DRAWINGS

FIG. 1 is a schematic diagram illustrating a cell culture apparatus in accordance with at least one example embodiment.

FIG. 2 is a block diagram of a culture control apparatus for use with the cell culture apparatus illustrated in FIG. 1 in accordance with at least one example embodiment.

FIG. 3 is a flowchart of illustrating an example method for cell culture processing using the cell culture apparatus illustrated in FIG. 1 in accordance with at least one example embodiment.

FIG. 4 is a schematic diagram illustrating a cell loading step using the cell culture apparatus illustrated in FIG. 1 in accordance with at least one example embodiment.

FIG. 5 is a schematic diagram illustrating a cell stirring step using the cell culture apparatus illustrated in FIG. 1 in accordance with at least one example embodiment.

FIG. 6 is a schematic diagram illustrating a cell packing step using the cell culture apparatus illustrated in FIG. 1 in accordance with at least one example embodiment.

FIG. 7 is a schematic diagram illustrating a cell culture step using the cell culture apparatus illustrated in FIG. 1 in accordance with at least one example embodiment.

FIG. 8 is a flowchart of the cell stirring step using the cell culture apparatus illustrated in FIG. 1 in accordance with at least one example embodiment.

FIGS. 9A and 9B are time charts illustrating a temporal change in the amount of received transmitted light detected by a sensor unit of the cell culture apparatus illustrated in FIG. 1 in accordance with at least one example embodiment.

### DETAILED DESCRIPTION

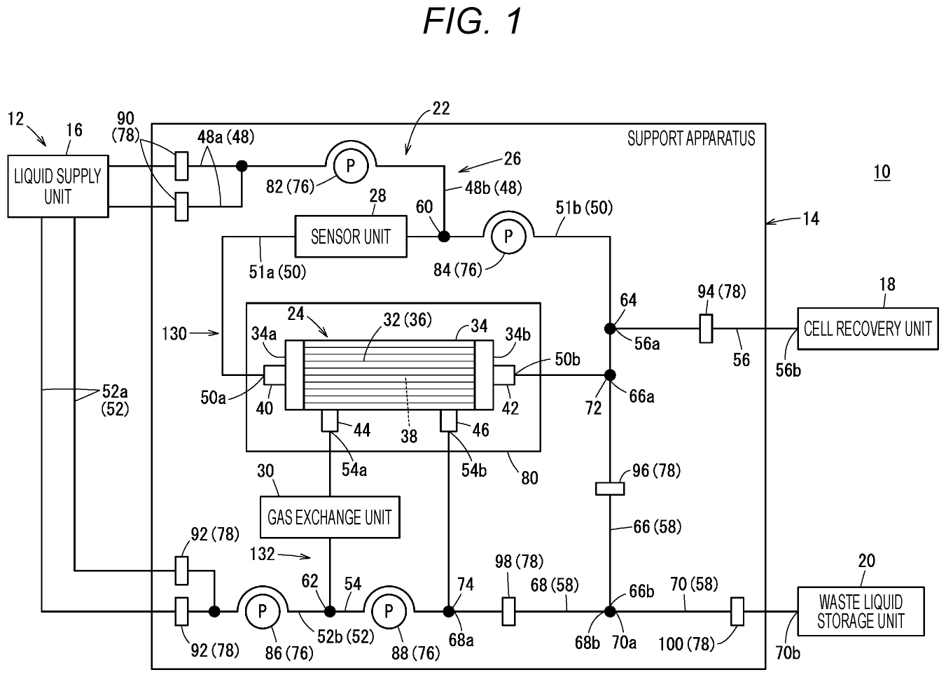

FIG. 1 is a schematic diagram illustrating a cell culture apparatus 10. The cell culture apparatus 10 may include a cell culture circuit 12 and a support apparatus 14. The cell culture apparatus 10 may be configured to culture cells as separated from biological tissue in a culture medium. The cells that are cultured using the cell culture apparatus 10 may include, for example, adherent cells, floating cells, or a combination of adherent cells and floating cells. For example, the cells that are cultured using the cell culture apparatus 10 may include embryonic stem(ES) cells, induced pluripotent stem (iPS) cells, mesenchymal stem cells, or any combination thereof.

Liquid flows through a cell culture circuit 12. The liquid may include a cell suspension, a culture medium, a washing solution, a dissociation solution, or any combination thereof. The cell suspension may be a solution containing cells. The culture medium may be a culture solution for growing cells. The culture medium may be selected according to the cell to be cultured. In at least one example embodiment, the culture medium may include minimum essential media. The washing solution may be a solution for washing the inside of the cell culture circuit 12. The washing solution may include, for example, water, a buffer solution, physiological saline, or any combination thereof. The buffer solution may include, for example, phosphate buffered salts (PBS), tris-buffered saline (TBS), or a combination of phosphate buffered salts and tris-buffered saline. The dissociation solution may be a solution for detaching cells from a bioreactor 24 of the cell culture circuit 12. The dissociation solution may include a trypsin solution, an EDTA solution (ethylenediaminetetraacetic acid solution), or a combination of trypsin solution and EDTA solution.

The cell culture circuit 12 may be a disposable product that can be replaced before each use of the cell culture apparatus 10. The cell culture circuit 12 may include a liquid supply unit 16, a cell recovery unit 18, a waste liquid storage unit 20, and a culture body 22.

The liquid supply unit 16 may include a plurality of medical bags (not illustrated). Each medical bag may be filled with a liquid to be supplied to the culture body 22. For example, in at least one example embodiment, the liquid supply unit 16 may include first medical bag that carries or supports a cell suspension; a second medical bag that carries or supports a culture medium; t a third medical bag that carries or supports a washing solution; a fourth medical bag that carries or supports a dissociation solution. Each of the plurality of medical bags may be individually filled.

Each of the cell recovery unit 18 and the waste liquid storage unit 20 may include a medical bag (not illustrated). The cell recovery unit 18 may be configured to recover the cells cultured in the culture body 22. The waste liquid storage unit 20 may be configured to store the waste liquid generated in the culture body 22.

The culture body 22 may include a bioreactor 24, a flow path 26, a sensor unit 28, and a gas exchange unit 30.

The bioreactor 24 may include a plurality of hollow fiber membranes 32 and a cylindrical housing 34. The plurality of hollow fiber membranes 32 may be stored in the housing 34. Each hollow fiber membrane 32 may extend along the longitudinal direction of the bioreactor 24. The hollow fiber membrane 32 may be made of, for example, a polymer material. The hollow fiber membrane 32 may have a plurality of pores (not illustrated). A first end portion of each hollow fiber membrane 32 may be fixed to a first end portion 34a in the longitudinal direction of the housing 34. The second end portion of each hollow fiber membrane 32 may be fixed to the second end portion 34b in the longitudinal direction of the housing 34.

The bioreactor 24 may include a first region 36 and a second region 38. The first region 36 may be a space inside each hollow fiber membrane 32. The second region 38 may be a space between the outer peripheral surface of each hollow fiber membrane 32 and the inner peripheral surface of the housing 34. The first region 36 and the second region 38 may communicate with each other through the plurality of pores of the hollow fiber membrane 32.

The housing 34 may include a first port 40, a second port 42, a third port 44, and a fourth port 46. The first port 40 may be disposed at the first end portion 34a of the housing 34. The first port 40 may be connected to a first end portion of each hollow fiber membrane 32. As a result, the first port 40 may communicate with the first region 36. The second port 42 may be disposed at the second end portion 34b of the housing 34. The second port 42 may be connected to the second end portion of each hollow fiber membrane 32. As a result, the second port 42 may communicate with the first region 36.

The third port 44 and the fourth port 46 may be disposed on the outer peripheral surface of the housing 34. The third port 44 may be disposed between the first port 40 and a central portion of the housing 34 in the longitudinal direction. The fourth port 46 may be disposed between the second port 42 and a central portion of the housing 34 in the longitudinal direction. The third port 44 and the fourth port 46 may both communicate with the second region 38.

The flow path 26 may include a plurality of tubes (pipes) through which liquid flows. Each tube may be made of a soft resin material. The flow path 26 may include a first supply flow path 48, a first circulation flow path 50, a second supply flow path 52, a second circulation flow path 54, a collection flow path 56, and a waste liquid flow path 58.

Via the first supply flow path 48, liquids from the liquid supply unit 16 may be introduce to the first circulation flow path 50. The first supply flow path 48 may include a plurality of first upstream flow paths 48a and one first downstream flow path 48b. For example, a first upstream flow path 48a may be provided for each medical bag of the liquid supply unit 16. Each of the first upstream flow paths 48a may be connected to at least one medical bag of the liquid supply unit 16. In addition, each of the first upstream flow paths 48a may be connected to the first downstream flow path 48b. The first downstream flow path 48b may be connected to a first merging portion 60 of the first circulation flow path 50.

The first circulation flow path 50 may introduce the liquid introduced from the first supply flow path 48 to the bioreactor 24. In addition, the first circulation flow path 50 may introduce the liquid discharged from the bioreactor 24 to the bioreactor 24 again. The first end portion 50a of the first circulation flow path 50 may be connected to the first port 40 of the bioreactor 24. The second end portion 50b of the first circulation flow path 50 may be connected to the second port 42 of the bioreactor 24. The first circulation flow path 50 may communicate with an inner hole of each hollow fiber membrane 32 (i.e., the first region 36). The first merging portion 60 may be disposed in the first circulation flow path 50. In the first circulation flow path 50, a portion partitioned by the first end portion 50a and the first merging portion 60 may be referred to as a first flow path 51a. In the first circulation flow path 50, a portion partitioned by the second end portion 50b and the first merging portion 60 may be referred to as a second flow path 51b. A collection branch portion 64 may be disposed in the second flow path 51b. Furthermore, in the second flow path 51b, a first branch portion 72 may be disposed between the collection branch portion 64 and the second end portion 50b.

Via the second supply flow path 52, liquids from the liquid supply unit 16 may be introduced to the second circulation flow path 54. The second supply flow path 52 may include a plurality of second upstream flow paths 52a and one second downstream flow path 52b. For example, one second upstream flow path 52a may be provided for each medical bag of the liquid supply unit 16. Each of the second upstream flow path 52a may be connected to at least one medical bag of the liquid supply unit 16. In addition, each of the second upstream flow paths 52a may be connected to the second downstream flow path 52b. The second downstream flow path 52b may be connected to a second merging portion 62 of the second circulation flow path 54.

The second circulation flow path 54 may introduce the liquid introduced from the second supply flow path 52 to the bioreactor 24. In addition, the second circulation flow path 54 may introduce the liquid discharged from the bioreactor 24 to the bioreactor 24 again. The first end portion 54a of the second circulation flow path 54 may be connected to the third port 44 of the bioreactor 24. A second end portion 54b of the second circulation flow path 54 may be connected to the fourth port 46 of the bioreactor 24. The second circulation flow path 54 may communicate with a space between the plurality of hollow fiber membranes 32 and the housing 34 (i.e., the second region 38). The second merging portion 62 may be disposed in the second circulation flow path 54. In addition, in the second circulation flow path 54, a second branch portion 74 may be disposed between the second merging portion 62 and the second end portion 54b.

The collection flow path 56 may introduce the cell suspension discharged from the bioreactor 24 to the cell recovery unit 18. The collection flow path 56 may branch from the first circulation flow path 50. A first end portion 56a of the collection flow path 56 may be connected to the collection branch portion 64 of the first circulation flow path 50. A second end portion 56b of the collection flow path 56 may be connected to the medical bag of the cell recovery unit 18.

The waste liquid flow path 58 may introduce the liquid in the first circulation flow path 50 and the second circulation flow path 54 to the waste liquid storage unit 20. The waste liquid flow path 58 may include a first waste liquid flow path 66, a second waste liquid flow path 68, and a third waste liquid flow path 70. The first waste liquid flow path 66 may branch from the first circulation flow path 50. A first end portion 66a of the first waste liquid flow path 66 may be connected to the first branch portion 72 of the first circulation flow path 50. The second waste liquid flow path 68 may branch from the second circulation flow path 54. A first end portion 68a of the second waste liquid flow path 68 may be connected to the second branch portion 74 of the second circulation flow path 54. Each of the second end portion 66b of the first waste liquid flow path 66 and the second end portion 68b of the second waste liquid flow path 68 may be connected to a first end portion 70a of the third waste liquid flow path 70. A second end portion 70b of the third waste liquid flow path 70 may be connected to the medical bag of the waste liquid storage unit 20.

The sensor unit 28 may be disposed in the first flow path 51a, for example, at a measurement target site. Although the first flow path 51a is discussed herein it should be appreciated that, in various other example embodiments, the sensor unit 28 may be instead disposed in the second flow path 51b. The sensor unit 28 may be configured to detect a predetermined or selected physical quantity related to a liquid (for example, a cell suspension) flowing through the first circulation flow path 50. The predetermined or selected physical quantity may be a physical quantity proportional to the number of cells in the liquid.

The sensor unit 28 may include a light source and one or more optical sensors (light receivers). The light source may irradiate the liquid with light. The optical sensor may receive transmitted light transmitted through the liquid. The optical sensor may output an electric signal corresponding to the amount of received light to a controller 112. The optical sensor may receive scattered light (forward scattered light, side scattered light, backscattered light, or a combination thereof in addition to or instead of the transmitted light. In at least one example embodiment, the sensor unit 28 may include a sensor (for example, a dielectric constant sensor) including two or more electrodes in addition to, or instead of, the light source and/or the optical sensor.

The gas exchange unit 30 may be disposed between the second merging portion 62 and the third port 44 in the second circulation flow path 54. The gas exchange unit 30 may be configured to supply a gas of a predetermined component to the liquid (culture medium) flowing through the second circulation flow path 54. The gas used in the gas exchange unit 30 may have a component close to air. For example, in at least one example embodiment, the gas may include nitrogen, oxygen, and carbon dioxide.

The cell culture circuit 12 may be detachable from the support apparatus 14. As illustrated in FIG. 1, a state in which the cell culture circuit 12 is attached to the support apparatus 14 may be referred to as a “set state”. The support apparatus 14 may include a cassette that supports the cell culture circuit 12. The support apparatus 14 may be a reusable product that can be used a plurality of times.

The support apparatus 14 may include a plurality of pumps 76, a plurality of clamps 78, and a reactor driving unit 80. Each of the plurality of pumps 76, the plurality of clamps 78, and the reactor driving unit 80 may include an electric actuator. Each of the plurality of pumps 76, the plurality of clamps 78, and the reactor driving unit 80 may include a fluid actuator.

Each pump 76 may be configured to apply a flow force to the liquid in the flow path 26, for example, by squeezing the tube forming the flow path 26. Each pump 76 may be operated by power supplied from a pump drive circuit 114.

The plurality of pumps 76 may include a first supply pump 82, a first circulation pump 84, a second supply pump 86, and a second circulation pump 88.

In the set state, the first downstream flow path 48b may be attached to the first supply pump 82. The first supply pump 82 may be configured to apply a flow force in a direction from the liquid supply unit 16 toward the first circulation flow path 50 to the liquid in the first supply flow path 48.

In the set state, the second flow path 51b of the first circulation flow path 50 may be attached to the first circulation pump 84. The first circulation pump 84 may be configured to apply a flow force in a direction from the second port 42 to the first port 40 to the liquid in the first circulation flow path 50. The first circulation pump 84 may also apply a flow force in a direction from the first port 40 to the second port 42 to the liquid in the first circulation flow path 50.

In the set state, the second downstream flow path 52b may be attached to the second supply pump 86. The second supply pump 86 may apply a flow force in a direction from the liquid supply unit 16 toward the second circulation flow path 54 to the liquid in the second supply flow path 52.

In the set state, the second circulation flow path 54 may be attached to the second circulation pump 88. The second circulation pump 88 may be configured to apply a flow force in a direction from the fourth port 46 to the third port 44 to the liquid in the second circulation flow path 54. The second circulation pump 88 may also apply a flow force in a direction from the third port 44 to the fourth port 46 to the liquid in the second circulation flow path 54.

Each clamp 78 may be configured to close the flow path 26 by compressing the tube forming the flow path 26 in the lateral direction. Each clamp 78 may thus function as an on-off valve. Each clamp 78 may be operated by power supplied from a clamp drive circuit 116.

The plurality of clamps 78 may include a plurality of first supply clamps 90, a plurality of second supply clamps 92, a collection clamp 94, a first waste liquid clamp 96, a second waste liquid clamp 98, and a third waste liquid clamp 100.

In the set state, one first upstream flow path 48a may be attached to one first supply clamp 90. In other words, each of the first upstream flow paths 48a may be supported by any one of the first supply clamps 90. The first supply clamp 90 may be configured to open and close the first supply flow path 48.

In the set state, one second upstream flow path 52a may be attached to one second supply clamp 92. In other words, each of the second upstream flow paths 52a may be supported by any one of the second supply clamps 92. The second supply clamp 92 may be configured to open and close the second supply flow path 52.

In the set state, the collection flow path 56 may be attached to the collection clamp 94. The collection clamp 94 may be configured to open and close the collection flow path 56. In the set state, the first waste liquid flow path 66 may be attached to the first waste liquid clamp 96. The first waste liquid clamp 96 may be configured to open and close the first waste liquid flow path 66. In the set state, the second waste liquid flow path 68 may be attached to the second waste liquid clamp 98. The second waste liquid clamp 98 may be configured to open and close the second waste liquid flow path 68. In the set state, the third waste liquid flow path 70 may be attached to the third waste liquid clamp 100. The third waste liquid clamp 100 may be configured to open and close the third waste liquid flow path 70.

The reactor driving unit 80 may be configured to support the bioreactor 24. For example, the reactor driving unit 80 may be configured to rotate the bioreactor 24 about an axis orthogonal to the longitudinal direction of the bioreactor 24. The reactor driving unit 80 may be operated by power supplied from a reactor drive circuit 118.

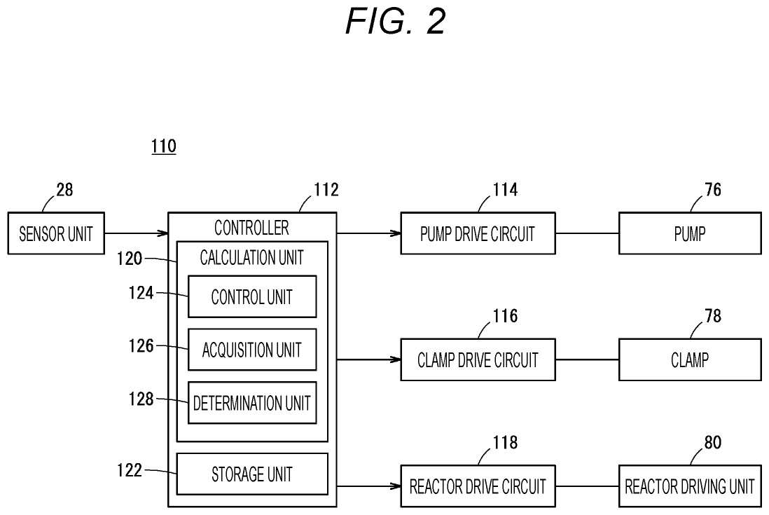

FIG. 2 is a block diagram of a culture control apparatus 110 included in the cell culture apparatus 10. The culture control apparatus 110 may be configured to control each of the plurality of pumps 76, each of the plurality of clamps 78, and the reactor driving unit 80. The culture control apparatus 110 may function as a stirring apparatus that stirs cell suspension held in or moving through the bioreactor 24.

The culture control apparatus 110 may include a sensor unit 28, a controller 112, a pump drive circuit 114, a clamp drive circuit 116, and a reactor drive circuit 118. The controller 112 may include a computer. The controller 112 may include a calculation unit 120 and a storage unit 122.

The calculation unit 120 may be configured by a processor (e.g., a central processing unit (CPU) and/or a graphics processing unit (GPU)). That is, the calculation unit 120 may be configured by a processing circuit.

The calculation unit 120 may include a control unit 124, an acquisition unit 126, and a determination unit 128. The control unit 124, the acquisition unit 126, and the determination unit 128 may each be realized by executing a program stored in the storage unit 122 by the calculation unit 120.

At least a part of the control unit 124 and/or at least part of the acquisition unit 126 and/or at least part of the determination unit 128 may be realized by an integrated circuit (e.g., as an application specific integrated circuit (ASIC) or a field-programmable gate array (FPGA)). At least a part of the control unit 124 and/or at least part of the acquisition unit 126 and/or at least part of the determination unit 128 may be configured by an electronic circuit including a discrete device.

The control unit 124 may be configured to output various operation signals and to control the operation of each pump 76, the operation of each clamp 78, and the operation of the reactor driving unit 80. The acquisition unit 126 may be configured to acquire the amount of received transmitted light (or the amount of received scattered light) over time on the basis of the electrical signal output from the sensor unit 28.

The storage unit 122 may include a volatile memory (not illustrated) and a non-volatile memory (not illustrated). The volatile memory may include, for example, a random access memory (RAM). The volatile memory may be used as a working memory of the processor and may be configured to temporarily store data and the like necessary for processing or operation. The non-volatile memory may include a read only memory (ROM) and a flash memory. The non-volatile memory may be used as a memory for storage and may be configured to store a program, a table, a map, and the like.

The pump drive circuit 114 may be configured to supply power to the actuator of the pump 76 to be operated according to a pump operation signal output from the controller 112. Each clamp drive circuit 116 may be configured to supply power to the actuator of the clamp 78 to be operated according to the clamp operation signal output from the controller 112. The reactor drive circuit 118 may be configured to supply power to the actuator of the reactor driving unit 80 in response to a reactor operation signal output from the controller 112.

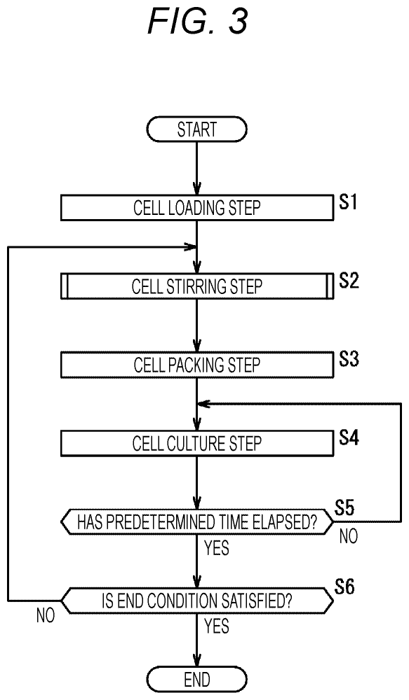

FIG. 3 is a flowchart illustrating an example method for cell culture processing. The user may use an input apparatus to perform an operation of starting the cell culture processing. The calculation unit 120 may be configured to start the cell culture processing illustrated in FIG. 3 in response to the start operation.

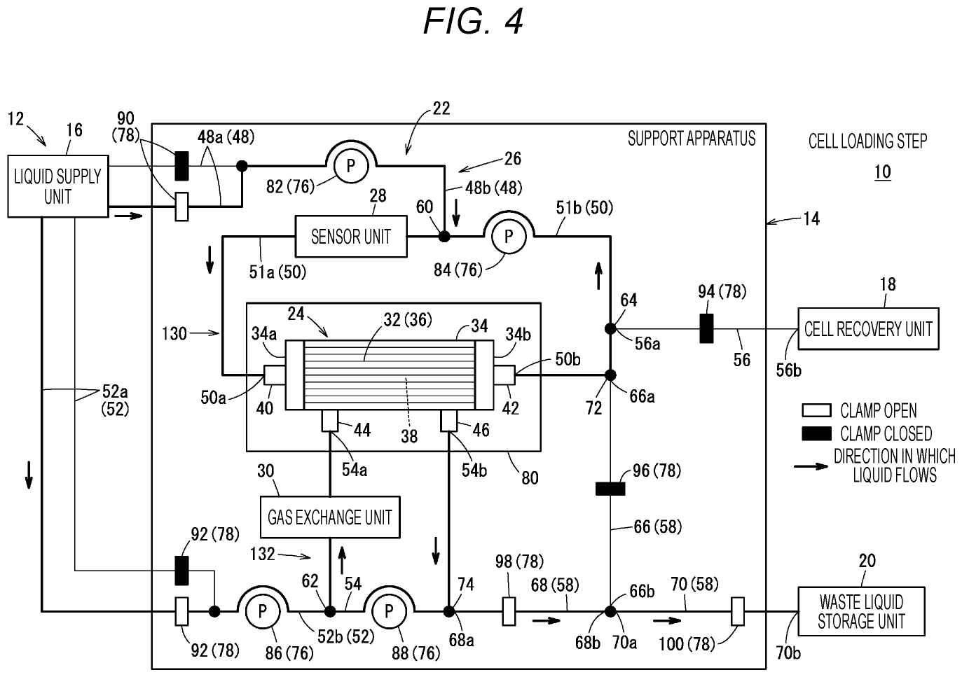

In step S1, the calculation unit 120 may be configured to performs a cell loading step. Here, the control unit 124 may configured to output a pump operation signal corresponding to the cell loading step to the pump drive circuit 114. In addition, the control unit 124 may be configured to output a clamp operation signal corresponding to the cell loading step to the clamp drive circuit 116. Then, the cell culture apparatus 10 may enter the state illustrated in FIG. 4. Eell suspension filled in the medical bag of the liquid supply unit 16 may flow through the first supply flow path 48 and the first circulation flow path 50 and may be supplied to the bioreactor 24. Thus, the cells may be introduced into the first region 36 (inside the hollow fiber membrane 32) of the bioreactor 24. The control unit 124 may be configured to stop the cell loading step at a predetermined or selected timing. When step S1 ends, the processing may proceed to step S2.

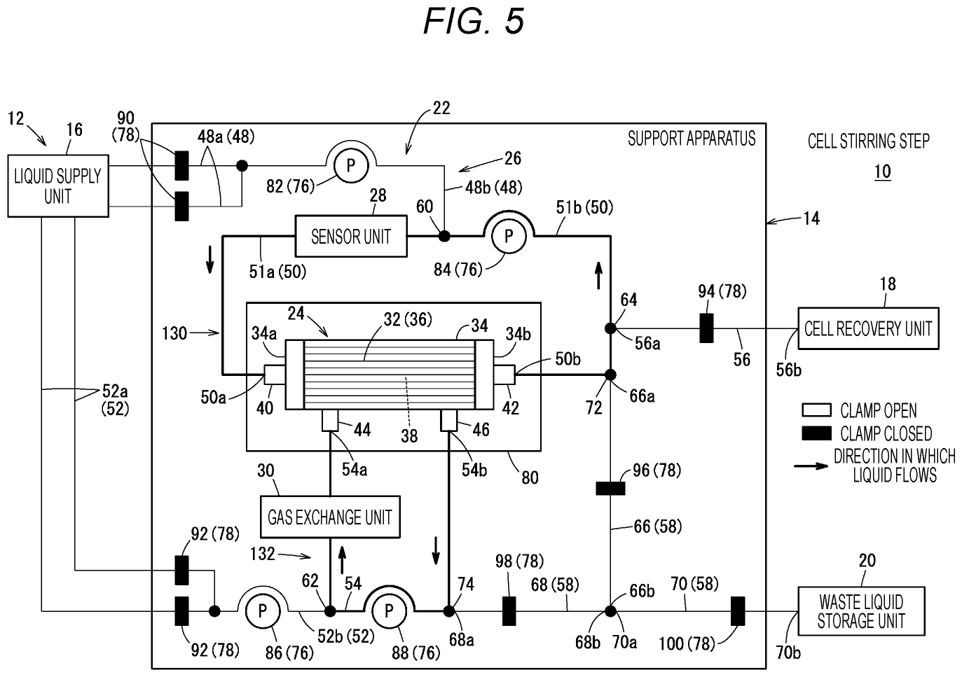

In step S2, the calculation unit 120 may be configured to perform a cell stirring step. Here, the control unit 124 may be configured to output a pump operation signal corresponding to the cell stirring step to the pump drive circuit 114. In addition, the control unit 124 may be configured to output a clamp operation signal corresponding to the cell stirring step to the clamp drive circuit 116. Then, the cell culture apparatus 10 may enter the state illustrated in FIG. 5. In the cell stirring step, the cell suspension may be stirred and the cells may be diffused into the cell suspension. A state in which cells are uniformly diffused in the cell suspension may be referred to as a uniform diffusion state. In a uniform diffusion state, nutrients are evenly distributed among the cells. Therefore, the uniform diffusion state can increase the growth rate of the cells. In addition, it may be possible to increase the survival rate of cells by setting the state to the uniform diffusion state. When step S2 ends, the processing may proceed to step S3.

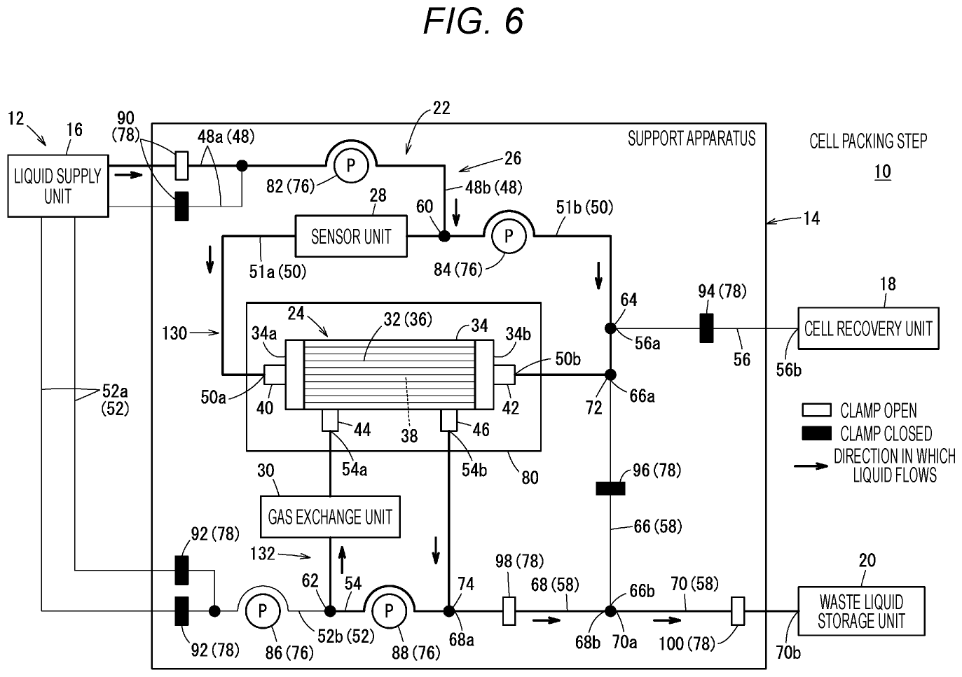

In step S3, the calculation unit 120 may be configured to perform a cell packing step. Here, the control unit 124 may be configured to output a pump operation signal corresponding to the cell packing step to the pump drive circuit 114. In addition, the control unit 124 may be configured to output a clamp operation signal corresponding to the cell packing step to the clamp drive circuit 116. Then, the cell culture apparatus 10 may enter the state illustrated in FIG. 6. The culture medium filled in the medical bag of the liquid supply unit 16 may flow through the first supply flow path 48 and the first flow path 51a and may be supplied to the bioreactor 24. In addition, the culture medium filled in the medical bag of the liquid supply unit 16 may flow through the first supply flow path 48 and the second flow path 51b and may be supplied to the bioreactor 24. As a result, the cells remaining in the first circulation flow path 50 may move to the first region 36 (inside the hollow fiber membrane 32) of the bioreactor 24. The supply amount of the culture medium from the first flow path 51a to the bioreactor 24 may be preferably equal to the supply amount of the culture medium from the second flow path 51b to the bioreactor 24. In addition, the culture medium may circulate in the circulation path 132 including the bioreactor 24 and the second circulation flow path 54. The excess culture medium may be discharged to the waste liquid storage unit 20. The control unit 124 may be configured to stop the cell packing step when a predetermined packing time has elapsed from the start of the cell packing step or when the supply amount of the culture medium reaches a predetermined or selected amount. When step S3 ends, the processing may proceed to step S4.

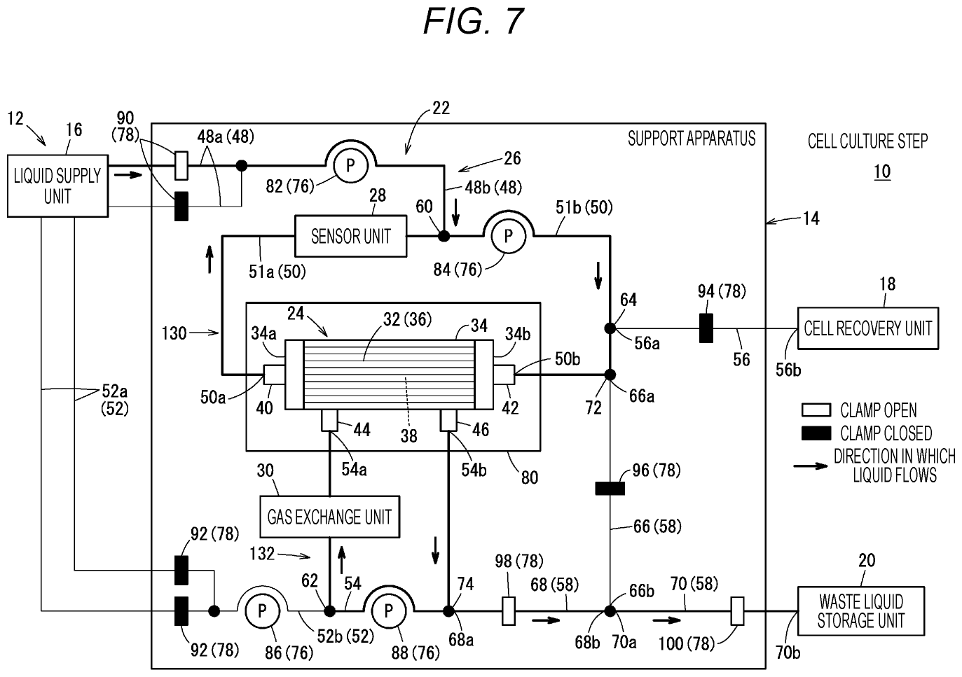

In step S4, the calculation unit 120 may be configured to perform a cell culture step. Here, the control unit 124 may be configured to output a pump operation signal corresponding to the cell culture step to the pump drive circuit 114. In addition, the control unit 124 may be configured to output a clamp operation signal corresponding to the cell culture step to the clamp drive circuit 116. Then, the cell culture apparatus 10 may enter the state illustrated in FIG. 7. The culture medium filled in the medical bag of the liquid supply unit 16 may flow through the first supply flow path 48 and the second flow path 51b and may be supplied to the first region 36 (inside the hollow fiber membrane 32) of the bioreactor 24. In addition, the culture medium may circulate in the circulation path 130 including the bioreactor 24 and the first circulation flow path 50 to provide nutrients to the cells. In addition, the culture medium may circulate in the circulation path 132 including the bioreactor 24 and the second circulation flow path 54. Excess culture medium may be discharged to the waste liquid storage unit 20.

In step S5, the determination unit 128 may be configured to determines whether a predetermined or selected culture time has elapsed since the start of the cell culture step. In at least one example embodiment, the storage unit 122 may be configured to store a predetermined time in advance. When the predetermined or selected time has elapsed (step S5: YES), the processing may proceed to step S6. On the other hand, when the predetermined or selected time has not elapsed (step S5: NO), the cell culture step of step S4 may be continuously performed.

When the processing proceeds from step S5 to step S6, the determination unit 128 may be configured to determine whether the end condition of the series of cell culture processing illustrated in FIG. 3 is satisfied. The storage unit 122 may be configured to store in advance a calibration curve indicating the relationship between the amount of received transmitted light (or scattered light) of the cell suspension and the number of cells. In addition, the storage unit 122 may be configured to store a threshold value as an end condition in advance. The determination unit 128 may be configured to estimate the number of cells contained in the cell suspension on the basis of the amount of light received by the sensor unit 28 and the calibration curve. The determination unit 128 may be configured to determine that the end condition is satisfied in a case where the estimated number of cells is equal to or greater than the threshold value. The storage unit 122 may be configured to store a calibration curve indicating the relationship between the amount of received transmitted light (or scattered light) of the cell suspension and the cell concentration. In a case where the estimated number of cells is equal to or larger than the threshold value (step S6: YES), the series of cell culture processing may be ended. On the other hand, in a case where the estimated number of cells is less than the threshold value (step S6: NO), the processing may return to the cell stirring step of step S2.

When the cell culture processing illustrated in FIG. 3 is completed, the control unit 124 may be configured to supply the dissociation solution to the bioreactor 24 and to transfer the cells inside the bioreactor 24 to the cell recovery unit 18.

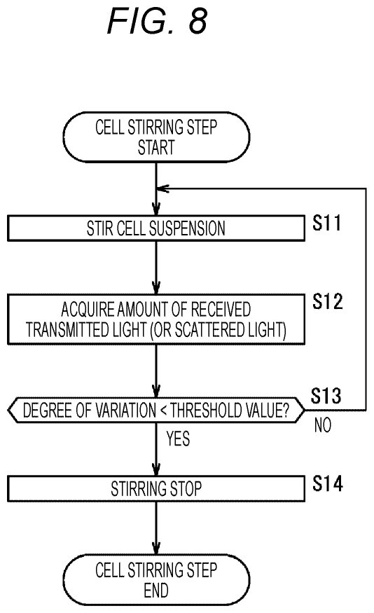

FIG. 8 is a flowchart of the cell stirring step. After step S1 illustrated in FIG. 3, or in a case where the determination is NO in step S6, the method may proceed to a cell stirring step.

In step S11 (cell stirring step), the control unit 124 may be configured to stir the cell suspension in the bioreactor 24. The control unit 124 may be configured to output a pump operation signal for operating the first circulation pump 84 to the pump drive circuit 114. The first circulation pump 84 may be configured to operate in response to the pump operation signal. As illustrated in FIG. 5, the cell suspension may circulate in a circulation path 130 defined by the bioreactor 24 and the first circulation flow path 50. Thereby, the cell suspension may be stirred. Further, the control unit 124 may be configured to output a reactor operation signal for operating the reactor driving unit 80 to the reactor drive circuit 118. The reactor driving unit 80 may be configured to rotate the bioreactor 24 in one direction and in the opposite direction according to the reactor operation signal, allowing the cell suspension to be homogeneously diffused more quickly. The processing of step S11 may be continued until the processing proceeds to step S14.

In step S12 (acquisition step), the acquisition unit 126 may be configured to acquire the amount of received transmitted light on the basis of the electric signal output by the sensor unit 28. The acquisition unit 126 may be configured to acquire the amount of received scattered light instead of the amount of received transmitted light. The acquisition unit 126 may be configured to acquire the amount of received transmitted light every predetermined or selected sampling time.

In step S13 (determination step), the determination unit 128 may be configured to compare the degree of variation of the received light amount of the transmitted light with the threshold value for the degree of variation. For example, the determination unit 128 may be configured to calculate a standard deviation of the amount of received transmitted light as the degree of variation. The storage unit 122 may be configured to store in advance a threshold value predetermined or selected for the degree of variation. As the threshold value, an upper limit value of an allowable degree of variation may be set. The determination unit 128 may be configured to calculate the standard deviation or variation of the received light amount of the transmitted light on the basis of the received light amount of the transmitted light for the most recent predetermined or selected number of times. The determination unit 128 may be configured to determine that the cells have reached the uniform diffusion state in the cell suspension on the basis of the fact that the standard deviation is smaller than the threshold value. When the standard deviation is smaller than the threshold value (step S13: YES), the processing may proceed to step S14. On the other hand, when the standard deviation is equal to or larger than the threshold value (step S13: NO), the processing may return to step S11 and the processing of step S11 may continue.

When the process proceeds from step S13 to step S14, the control unit 124 may be configured to stop stirring the cell suspension. That is, the control unit 124 may be configured to stop the first circulation pump 84. In addition, the control unit 124 may also be configured to stop the rotation of the bioreactor 24. When step S14 ends, the processing may proceed to the cell packing step (step S3).

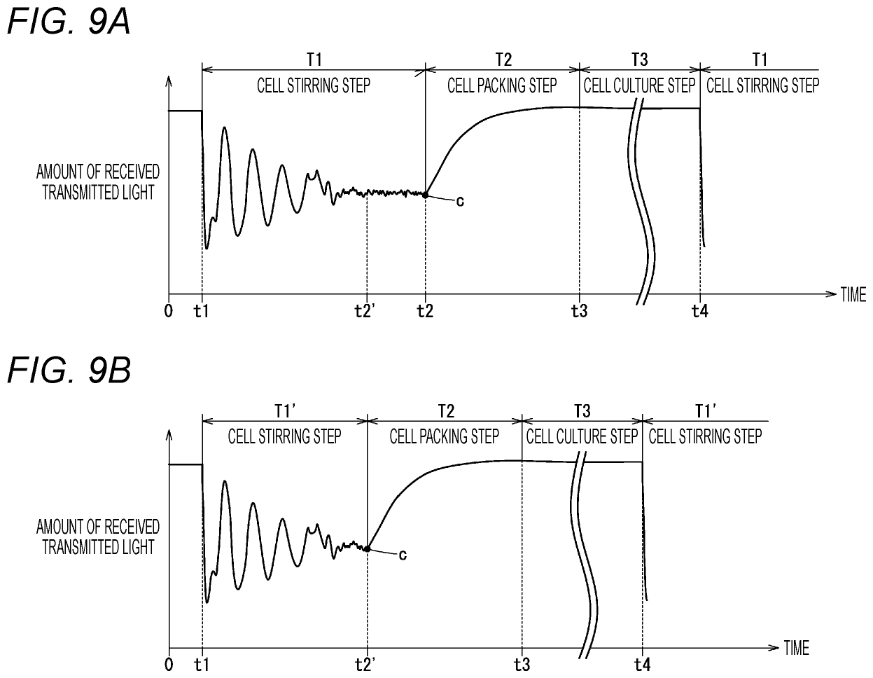

FIGS. 9A and 9B are time charts illustrating temporal changes in the amount of received transmitted light detected by the sensor unit 28. FIG. 9A illustrates a temporal change in the amount of received transmitted light in the cell culture processing without using the culture control apparatus 110. FIG. 9B illustrates a temporal change in the amount of received transmitted light in the cell culture processing using the culture control apparatus 110. In the cell culture processing illustrated in FIG. 9A, the time T1 from the time point t1 to the time point t2 is the execution time of the cell stirring step. In the cell culture processing illustrated in FIG. 9A, the time T2 from the time point t2 to the time point t3 is the execution time of the cell packing step. In the cell culture processing illustrated in FIG. 9A, the time T3 from the time point t3 to the time point t4 is the execution time of the cell culture step.

In the cell stirring step illustrated in FIG. 9A, most of the cells are inside the bioreactor 24 at a stage before stirring (before time point t1). Therefore, the culture medium flowing through the first circulation flow path 50 contains few cells. Therefore, the turbidity of the liquid flowing through the first circulation flow path 50 is low and the amount of received transmitted light detected by the sensor unit 28 is large. When stirring is started at time point t1, cells inside the bioreactor 24 flow through the first circulation flow path 50. Then, a portion having a high cell concentration and a portion having a low cell concentration alternately appear in the liquid flowing through the first circulation flow path 50. As a result, the amount of received transmitted light detected by the sensor unit 28 changes with the lapse of time. When the stirring is continued, the received light amount of the transmitted light detected by the sensor unit 28 gradually converges to a substantially constant value c. The fact that the received light amount of the transmitted light detected by the sensor unit 28 converges to the constant value c means that the cells are uniformly diffused in the liquid (cell suspension). The cell stirring step illustrated in FIG. 9B has already converged to the constant value c at a time point t2′ earlier than the time point t2. That is, the cell stirring step performed from the time point t2′ to the time point t2 is unnecessary.

The determination unit 128 is configured to compare the standard deviation (i.e., degree of variation) with a threshold value (i.e., predetermined or selected degree) (see step S13 in FIG. 8). As a result, the determination unit 128 can determine that the uniform diffusion state has been reached, allowing the cell stirring step to be executed only for a necessary minimum time. The execution time (time T1′) of the cell stirring step can be shortened, and the execution time of the cell culture processing illustrated in FIG. 3 can be shortened. Accordingly, the cell culture processing can be optimized.

The present invention is not limited to the above disclosure. Various configurations can be adopted without departing from the gist of the present invention.

1. A stirring method comprising:
stirring a cell suspension in a circulation path that includes a bioreactor by rotating the bioreactor;
acquiring a physical quantity related to the cell suspension in the circulation path; and
using the acquired physical quantity, determining if the cell suspension has reached a uniform diffusion state where cells are uniformly diffused in the cell suspension.

2. The stirring method of claim 1, wherein the physical quantity related to the cell suspension is acquired at a predetermined measurement target site.

3. The stirring method of claim 1, wherein the physical quantity related to the cell suspension is acquired over a period of time.

4. The stirring method of claim 1, wherein the uniform diffusion state has been reached when a degree of variation for the physical quantity is different than a predetermined or selected degree.

5. The stirring method of claim 4, wherein the uniform diffusion state has been reached when the degree of variation for the physical quantity is less than the predetermined or selected degree.

6. The stirring method of claim 4, wherein the degree of variation of the physical quantity is a standard deviation of a plurality of the physical quantity within a predetermined or selected unit of time.

7. The stirring method of claim 1, wherein the physical quantity is acquired using an optical sensor.

8. The stirring method of claim 7, wherein the physical quantity is at least one of an amount of transmitted light transmitted through the cell suspension and an amount of scattered light scattered by the cell suspension.

9. The stirring method of claim 1, further comprising:
stopping the stirring of the cell suspension when it is determined that the uniform diffusion state has been reached.

10. (canceled)

11. A stirring apparatus comprising:
a control unit configured to initiate stirring of a cell suspension in a circulation path that includes a bioreactor by rotating the bioreactor;
an acquisition unit configured to acquire a physical quantity related to the cell suspension in the circulation path; and
a determination unit configured to determine if the cell suspension has reached a uniform diffusion state where cells are uniformly diffused in the cell suspension.

12. The stirring apparatus of claim 11, wherein the acquisition unit is configured to acquire the physical quantity related to the cell suspension at a predetermined measurement target site.

13. The stirring apparatus of claim 11, wherein the acquisition unit is configured to acquire the physical quantity related to the cell suspension over a period of time.

14. The stirring apparatus of claim 11, wherein the determination unit is configured to determine that the uniform diffusion state has been reached when a degree of variation for the physical quantity is different than a predetermined or selected degree.

15. The stirring apparatus of claim 14, wherein determination unit is configured to determine that the uniform diffusion state has been reached when the degree of variation for the physical quantity is less than the predetermined or selected degree.

16. The stirring apparatus of claim 14, wherein the degree of variation of the physical quantity is a standard deviation of a plurality of the physical quantity within a predetermined or selected unit of time.

17. The stirring apparatus of claim 11, wherein the physical quantity is acquired using an optical sensor.

18. The stirring apparatus of claim 17, wherein the physical quantity is at least one of an amount of transmitted light transmitted through the cell suspension and an amount of scattered light scattered by the cell suspension.

19. The stirring apparatus of claim 17, wherein the control unit is further configured to stop the stirring of the cell suspension when the determination unit has determined that the uniform diffusion state has been reached.

20. (canceled)
